(19) United States
(12) Patent Application Publication
(10) Pub. No.: US 2006/0275367 A1
(43) Pub. Date: Dec. 7, 2006

(54) Extended release formulations

(76) Inventor: Shubha Chungi, Chestnut Hill, MA (US)

Correspondence Address: GOODWIN PROCTER LLP;PATENT ADMINISTRATOR, EXCHANGE PLACE, BOSTON, MA, 02109-2881, US

(21) Appl. No.: 11/410,502

(22) Filed: Apr. 25, 2006

Related U.S. Application Data
(60) Provisional application No. 60/674,444, filed on Apr. 25, 2005.

(51) Int. Cl. A61K 31/55; A61K 9/26

(52) U.S. Cl. 424/469; 514/217

(57) ABSTRACT

The present invention relates to an extended release formulation containing a poorly water soluble active ingredient and to a method for preparing the formulation. The formulation contains a wax-based extended release material, which provides the extended release of the active ingredient.

### RELATED APPLICATIONS

This application claims the benefit of and priority to U.S. patent application Ser. No. 60/674,444, filed on Apr. 25, 2005, the disclosure of which is incorporated by reference herein.

### FIELD OF INVENTION

The invention relates generally to extended release formulations containing a poorly water soluble active ingredient and, more particularly, relates to wax-based formulations containing a poorly water soluble active ingredient.

### BACKGROUND OF THE INVENTION

It is well known that differences in chemical or physical properties of pharmaceuticals, such as solubility, can affect their bioavailability and effective clinical use (J. Pharm. Sci., 58:911-929 (1969)). While many extended release formulations are already known, certain poorly water soluble active ingredients present formulation difficulties which render them inapplicable for extended release formulations that might be suitable for, e.g., relatively soluble active ingredients.

Dissolving poorly water soluble active ingredients into aqueous solutions appropriate for human use (e.g., oral, topical application, intravenous injection, intramuscular injection, subcutaneous injection) have resulted in a number of serious side effects caused not by the active ingredients but by the carrier agents used to dissolve the active ingredients. Clearly, an approach aimed at providing extended release formulations of these active ingredients and avoiding the complications of solubilizing agents would enhance the quality of health care to patients.

For example, carbamazepine is a well-known poorly water soluble pharmaceutical agent for the clinical treatment of seizure disorders, including tonic-clonic seizures, complex partial seizures and trigeminal neuralgia. In the presence of water, carbamazepine, which is hydrophobic, is known to transform rapidly to carbamazepine dihydrate crystals. Carbamazepine often exhibits poor bioavailability when incorporated into extended release formulations.

Because there is a correlation between peak concentrations of carbamazepine and central nervous system (CNS) side effects, especially in patients receiving polytherapy (Epilepsia, 28:507-514 (1987); Epilepsia, 28:286-299 (1987); Epilepsia, 21:341-350 (1980); Epilepsia, 25:476-481 (1984) and Arch. Neurol., 41:830-834 (1984)), it is of great clinical importance to assure a steady level of carbamazepine during a relatively long period of time. Currently, however, there are a limited number of oral therapeutic systems containing carbamazepine in extended release form.

Many of the available extended release compositions for poorly water soluble active ingredients also have the inherent drawbacks of being expensive and require time-consuming methods of production. Accordingly, there is an ongoing need for additional extended release compositions for poorly water soluble active ingredients.

### SUMMARY OF THE INVENTION

The present invention provides an extended release formulation, as well as a simple and easy method to prepare such an extended release formulation that can be employed for a poorly water soluble active ingredient. The formulation contains a wax-based extended release material. The formulation may be prepared, for example, by simple granulation methods, e.g., hot melt granulation.

The present invention provides an extended release formulation comprising a plurality of granules comprising a poorly water soluble active ingredient and a wax-based extended release material, wherein the plurality of granules, when characterized using sieve analysis, have an average size from about 10 mesh to about 100 mesh, preferably from about 10 mesh to about 80 mesh, and more preferably from about 16 mesh to about 70 mesh.

The present invention also provides an extended release formulation comprising a plurality of granules comprising a poorly water soluble active ingredient and a wax-based extended release material, such that, when characterized by sieve analysis, about 40% to 80% of the granules are retained on a 60 mesh screen. Preferably, about 55% to about 75% of the granules are retained on a 60 mesh screen. More preferably, about 60% to about 70% of the granules are retained on a 60 mesh screen.

The present invention also provide an extended release formulation comprising a plurality of granules comprising carbamazepine and a wax-based extended release material, wherein the extended release formulation has an in vitro dissolution profile, when measured using a USP apparatus II at 50 rpm in 1,000 mL dissolution medium (pH 1.2 and then pH 6.8) at 37° C., such that at least from about 60% to 75% (by wt) active ingredient is released after 2 hours, at least about from about 75% to 90% (by wt) active ingredient is released after 4 hours, at least about from about 85% to 100% (by wt) active ingredient is released after 8 hours. Preferably, the in vitro release profile is chosen such that the peak plasma level of carbamazepine obtained in vivo occurs at least 15 hours after administration.

Preferably, the active ingredient is selected from the group consisting of carbamazepine, phendimetrazine tartrate, indomethacin, disopyramide phosphate, and ketoprofen. More preferably, the active ingredient is carbamazepine.

In one embodiment, the wax-based extended release material is carnauba wax, bees wax or a combination thereof. Preferably, the wax-based extended release material is present in an amount of from about 1% to about 50% by weight of total weight of the granules. More preferably, the wax-based extended release material is present in an amount of from about 5% to about 25% by weight of total weight of the granules. Yet more preferably, the wax-based extended release material is present in an amount of from about 8% to about 16% by weight of total weight of the granules.

The formulation of the present invention may further comprise one or more inert additives selected from the group consisting of a wetting agent, a filler, a binder, and a surfactant.

The present invention provides a method for preparing an extended release formulation containing a poorly water soluble active ingredient, the method comprising: (a) melting a wax-based extended release material; and (b) mixing the active ingredient with the melted wax-based extended release material at a temperature higher than the melting temperature of the wax-based extended release material to produce the extended release formulation.

The method may further comprise the step of (c) performing sieve analysis to select granules having an average size from about 10 mesh to about 100 mesh to produce the extended release formulation. Preferably, granules are selected to have an average size from about 10 mesh to about 80 mesh. More preferably, granules are selected to have an average size from about 16 mesh to about 70 mesh.

The method may further comprise the step of (c) performing sieve analysis to select granules such that from about 40% to 80% of the granules are retained on a 60 mesh sieve to produce the extended release formulation. Preferably, granules are selected such that from about 55% to 75% of the granules are retained on a 60 mesh sieve. More preferably, granules are selected such that from about 60% to 70% of the granules are retained on a 60 mesh sieve.

The method may also comprise admixing one or more inert additives with the melted wax-based extended release material.

### BRIEF DESCRIPTION OF DRAWINGS

The objects and features of the invention may be better understood by reference to the drawing described below in which,

FIG. 1 illustrates a normalized dissolution profile comparison between Carbatrol® and four carbamazepine formulation batches prepared according to one embodiment of the invention.

### DETAILED DESCRIPTION OF THE INVENTION

The present invention relates to extended release formulations containing a poorly water soluble active ingredient and a wax-based extended release material, where the wax-based extended release material provides the extended release of the active ingredient. Such formulations may be formed, for example, by hot melt granulation.

The term “poorly water soluble active ingredients” refers to compounds having solubility in water of no more than about 20 mg/mL at physiological pH (6.5-7.4) at or below normal physiological temperatures. Preferably their water solubility is no more than about 10 mg/mL, more preferably no more than about 5 mg/mL, more preferably no more than about 0.5 mg/mL and even more preferably no more than about 0.05 mg/mL. Solubility of a variety of active ingredients are known in the art, for example, in U.S. Pharmacopeia (USP) (2003), Physician Desk Reference (PDR, 2005), or The Merck Index (the 13^{th} Ed. 2001).

Poorly water soluble active ingredients contemplated for use in the practice of the present invention include, but are not limited to:

Anti-convulsants (e.g., valproic acid, phenytoin, clonazepam, primidone, phenobarbitol, carbamazepine, amobarbital sodium, methsuximide, metharbital, mephobarbital, mephenytoin, phensuximide, paramethadione, ethotoin, phenacemide, secobarbitol sodium, clorazepate dipotassium, trimethadione, and the like);

Analgesics/antipyretics (e.g., ibuprofen, buprenorphine hydrochloride, propoxyphene napsylate, levorphanol tartrate, diflunisal, trolamine salicylate, mefenamic acid, butorphanol tartrate, choline salicylate, butalbital, phenyltoloxamine citrate, methotrimeprazine, meprobamate, and the like);

Anesthetics (e.g., halothane, isoflurane, methoxyflurane, propofol, thiobarbiturates, xenon and the like);

Anti-asthmatics (e.g., Azelastine, Ketotifen, Traxanox, and the like);

Antibiotics (e.g., neomycin, streptomycin, chloramphenicol, cephalosporin, ampicillin, penicillin, tetracycline, and the like);

Anti-depressants (e.g., nefopam, oxypertine, amoxapine, trazodone hydrochloride, maprotiline hydrochloride, phenelzine sulfate, desipramine hydrochloride, nortriptyline hydrochloride, tranylcypromine sulfate, imipramine pamoate, nortriptyline, isocarboxazid, desipramine hydrochloride, trimipramine maleate, protriptyline hydrochloride, and the like);

Anti-diabetics (e.g., biguanides, hormones, sulfonylurea derivatives, and the like);

Anti-fungal agents (e.g., griseofulvin, keoconazole, amphotericin B, Nystatin, candicidin, and the like);

Anti-hypertensive agents (e.g., propanolol, propafenone, oxyprenolol, nifedipine, reserpine, trimethaphan camsylate, phenoxybenzamine hydrochloride, pargyline hydrochloride, deserpidine, diazoxide, guanethidine monosulfate, minoxidil, rescinamine, sodium nitroprusside, rauwolfia serpentina, alseroxylon, phentolamine mesylate, reserpine, and the like);

Anti-inflammatories (e.g., (non-steroidal) indomethacin, naproxen, ibuprofen, ramifenazone, piroxicam, (steroidal) cortisone, dexamethasone, fluazacort, hydrocortisone, prednisolone, prednisone, and the like);

Anti-neoplastics (e.g., adriamycin, cyclophosphamide, actinomycin, bleomycin, duanorubicin, doxorubicin, epirubicin, mitomycin, methotrexate, fluorouracil, carboplatin, carmustine (BCNU), methyl-CCNU, cisplatin, etoposide, interferons, camptothecin and derivatives thereof, phenesterine, taxol and derivatives thereof, taxotere and derivatives thereof, vinblastine, vincristine, tamoxifen, etoposide, piposulfan, and the like);

Anti-anxiety agents (e.g., lorazepam, prazepam, chlordiazepoxide hydrochloride, oxazepam, clorazepate dipotassium, diazepam, hydroxyzine pamoate, hydroxyzine hydrochloride, alprazolam, droperidol, halazepam, chlormezanone, dantrolene, and the like);

Immunosuppressive agents (e.g., cyclosporine, azathioprine, mizoribine, FK506 (tacrolimus), and the like); antimigraine agents (e.g., ergotamine tartrate, propanolol hydrochloride, isometheptene mucate, dichloralphenazone, and the like);

Sedatives/hypnotics (e.g., barbiturates (e.g., pentobarbital, pentobarbital sodium, secobarbital sodium), benzodiazapines (e.g., flurazepam hydrochloride, triazolam, tomazeparm, midazolam hydrochloride, and the like);

Anti-anginal agents (e.g., beta-adrenergic blockers, calcium channel blockers (e.g., nifedipine, and the like), nitrates (e.g., nitroglycerin, isosorbide dinitrate, pentaerythritol tetranitrate, erythrityl tetranitrate, and the like));

Anti-psychotic agents (e.g., haloperidol, loxapine succinate, thioridazine, thiothixene, fluphenazine hydrochloride, fluphenazine enanthate, trifluoperazine hydrochloride, chlorpromazine hydrochloride, perphenazine, prochlorperazine, and the like);

Anti-manic agents (e.g., lithium carbonate);

Anti-arrhythmics (e.g., amiodarone, related derivatives of amiodarone, bretylium tosylate, esmolol hydrochloride, encainide hydrochloride, digoxin, digitoxin, mexiletine hydrochloride, disopyramide phosphate, quinidine gluconate, quinidine polygalacturonate, flecainide acetate, tocainide hydrochloride, and the like);

Anti-arthritic agents (e.g., phenylbutazone, sulindac, penicillamine, salsalate, piroxicam, azathioprine, indomethacin, meclofenamate sodium, gold sodium thiomalate, ketoprofen, auranofin, aurothioglucose, and the like);

Anti-gout agents (e.g., colchicine, allopurinol, and the like);

Anti-coagulants (e.g., heparin, heparin sodium, warfarin sodium, and the like);

Thrombolytic agents (e.g., urokinase, streptokinase, altoplase, and the like);

Anti-fibrinolytic agents (e.g., aminocaproic acid, and the like);

Hemorheologic agents (e.g., pentoxifylline, and the like);

Anti-platelet agents (e.g., aspirin, empirin, ascriptin, and the like);

Appetite suppressant (e.g., phendimetrazine tartrate, and the like);

Anti-parkinson agents (e.g., ethosuximide, and the like);

Anti-histamines/anti-pruritics (e.g., hydroxyzine hydrochloride, cyproheptadine hydrochloride, terfenadine, triprolidine hydrochloride, carbinoxamine maleate, diphenylpyraline hydrochloride, phenindamine tartrate, azatadine maleate, tripelennamine hydrochloride, dexchlorpheniramine maleate, methdilazine hydrochloride, trimprazine tartrate, and the like);

Agents useful for calcium regulation (e.g., calcitonin, parathyroid hormone, and the like);

Antibacterial agents (e.g., amikacin sulfate, aztreonam, chloramphenicol, chloramphenicol palmitate, chloramphenicol sodium succinate, ciprofloxacin hydrochloride, clindamycin hydrochloride, clindamycin palmitate, clindamycin phosphate, metronidazole, metronidazole hydrochloride, gentamicin sulfate, lincomycin hydrochloride, polymyxin B sulfate, colistimethate sodium, colistin sulfate, and the like);

Antiviral agents (e.g., interferon gamma, zidovudine, ribavirin, acyclovir, and the like);

Anti-microbials (e.g., cephalosporins (e.g., cefazolin sodium, cephradine, cefaclor, cephapirin sodium, ceftizoxime sodium, cefoperazone sodium, cefotetan disodium, cefutoxime azotil, cefotaxime sodium, cefadroxil monohydrate, ceftazidime, cephalexin, cephalothin sodium, cephalexin hydrochloride monohydrate, cefamandole nafate, cefoxitin sodium, cefonicid sodium, ceforanide, ceftriaxone sodium, ceftazidime, cefadroxil, cephradine, cefuroxime sodium, and the like), prythronycins, penicillins (e.g., ampicillin, amoxicillin, penicillin G benzathine, cyclacillin, ampicillin sodium, penicillin G potassium, penicillin V potassium, piperacillin sodium, oxacillin sodium, bacampicillin hydrochloride, cloxacillin sodium, ticarcillin disodium, azlocillin sodium, carbenicillin indanyl sodium, penicillin G potassium, penicillin G procaine, methicillin sodium, nafcillin sodium, and the like), erythromycins (e.g., erythromycin ethylsuccinate, erythromycin, erythromycin estolate, erythromycin lactobionate, erythromycin siearate, erythromycin ethylsuccinate, and the like), tetracyclines (e.g., minocycline hydrochloride, and the like), and the like);

Anti-infectives (e.g., GM-CSF);

Bronchodilators (e.g., sympathomimetics (e.g., epinephrine hydrochloride, metaproterenol sulfate, terbutaline sulfate, isoetharine, isoetharine mesylate, isoetharine hydrochloride, albuterol, bitolterol, mesylate isoproterenol hydrochloride, terbutaline sulfate, epinephrine bitartrate, epinephrine,), anticholinergic agents (e.g., ipratropium bromide), xanthines (e.g., aminophylline, dyphylline, aminophylline), mast cell stabilizers, inhalant corticosteroids (e.g., flurisolidebeclomethasone dipropionate, beclomethasone dipropionate monohydrate), salbutamol, beclomethasone dipropionate (BDP), ipratropium bromide, budesonide, ketotifen, salmeterol, xinafoate, terbutaline sulfate, triamcinolone, theophylline, nedocromil sodium, metaproterenol sulfate, albuterol, flunisolide, and the like);

Hormones (e.g., androgens (e.g., danazol, testosterone cypionate, fluoxymesterone, ethyltostosterone, testosterone enanihate, methyltestosterone, fluoxymesterone, testosterone cypionate), estrogens (e.g., estradiol, estropipate, conjugated estrogens), progestins (e.g., methoxyprogesterone acetate, norethindrone acetate), corticosteroids (e.g., triamcinolone, betamethasone, betamethasone sodium phosphate, dexamethasone, dexamethasone sodium phosphate, dexamethasone acetate, prednisone, methylprednisolone acetate suspension, triamcinolone acetonide, methylprednisolone, prednisolone sodium phosphate methylprednisolone sodium succinate, hydrocortisone sodium succinate, methylprednisolone sodium succinate, triamcinolone hexacatonide, hydrocortisone, hydrocortisone cypionate, prednisolone, fluorocortisone acetate, paramethasone acetate, prednisolone tebulate, prednisolone acetate, prednisolone sodium phosphate, hydrocortisone sodium succinate, and the like), thyroid hormones (e.g., levothyroxine sodium and the like);

Hypoglycemic agents (e.g., human insulin, purified beef insulin, purified pork insulin, glyburide, chlorpropamide, glipizide, tolbutamide, tolazamide, and the like);

Hypolipidemic agents (e.g., clofibrate, dextrothyroxine sodium, probucol, lovastatin, niacin, and the like);

Proteins (e.g., DNase, alginase, superoxide dismutase, lipase, and the like);

Nucleic acids (e.g., sense or anti-sense nucleic acids encoding any therapeutically useful protein, including any of the proteins described herein, and the like);

Agents useful for erythropoiesis stimulation (e.g., erythropoietin, and the like);

Anti-ulcer/anti-reflux agents (e.g., famotidine, cimetidine, ranitidine hydrochloride, and the like);

Anti-nauseants/anti-emetics (e.g., meclizine hydrochloride, nabilone, prochlorperazine, dimenhydrinate, promethazine hydrochloride, thiethylperazine, scopolamine, and the like);

Oil-soluble vitamins (e.g., vitamins A, D, E, K, and the like); and

Other active ingredients such as mitotane, visadine, halonitrosoureas, anthrocyclines, ellipticine, and the like.

Preferred poorly water soluble active ingredients include anticonvulsants. More preferably, the poorly water soluble anticonvulsant is valproic acid, divalproate sodium, phenytoin, phenytoin sodium, clonazepam, primidone, phenobarbitol, phenobarbitol sodium, carbamazepine, amobarbital sodium, methsuximide, metharbital, mephobarbital, mephenytoin, phensuximide, paramethadione, ethotoin, phenacemide, secobarbitol sodium, clorazepate dipotassium, or trimethadione. In one embodiment of the present invention, the active ingredient is carbamazepine. In another embodiment of the present invention, the active ingredient is phendimetrazine tartrate, indomethacin, phenytoin sodium, disopyramide phosphate, or ketoprofen.

The extended release formulation of the present invention may contain a single active ingredient, or two or more active ingredients. When two or more active ingredients are included, they can be mixed together or be in the form of a multiple unit dosage form, including a multi-layer dosage form, as known in the art.

The term “extended release” refers to the fact that the active ingredient is released from the formulation at an extended rate such that therapeutically beneficial blood levels of the active ingredient are maintained over a prolonged period of time, e.g., providing an about 8 to 24 hour dosage form. The term “extended release,” includes “controlled release,” “sustained release,” “prolonged release,” and other similar terms used in the art which provide a benefit as defined above. An “extended release material” is a substance, typically a polymer, that provides an extended release.

A wax-based extended release material provides the desired extended release for the formulations of the present invention. Wax-based extended release materials, according to the present invention include, but are not limited to, waxes of the animal or vegetable origin, synthetic waxes and semi-synthetic waxes. Preferably, no other extended release polymers (such as hydrophilic or hydrophobic cellulosic polymers) are used in addition to the wax-based extended release material.

The wax-based extended release material of the invention may have a melting range between about 40° C. and about 120° C. and, therefore, is solid at room temperature. Such waxes include, but are not limited to, higher fatty acids, higher fatty acid ester derivatives, higher alcohols and higher alcohol ester derivatives, among others, can also be used. Examples include:

Higher fatty acids (lauric acid, tridecanoic acid, myristic acid, pentadecanoic acid, palmitic acid, margaric acid, stearic acid, nonadecanoic acid, arachidonic acid, behenic acid, lignoceric acid, cerotic acid, montanic acid);

Higher fatty acid ester derivatives (glyceryl, ethylene glycol, propylene glycol, sorbitol, polyethylene glycol and other esters of the higher fatty acids listed above; saturated fatty acid glycerides derived from animals or vegetable, mixtures thereof, and hydrogenated oils available from said glycerides of the animal or vegitable origin; glycerides of oleic acid, linolic acid, linolenic acid, ricinoleic acid, etc. and mixtures thereof);

Higher alcohols (pentadecanol, hexadecanol, heptadecanol, octadecanol, nonadecanol, eicosanol, wool alcohol, cholesterol); and

Higher alcohol ester derivatives (cholesteryl palmitate and phytosterol palmitate).

Preferred wax-based extended release materials of the invention include high temperature melting, pharmaceutically acceptable water-insoluble wax or waxy substance, such as a saturated fat, for example, carnauba wax, white wax, bees wax, glyceryl monostearate, glyceryl oleate, paraffin or spermaceti. In one embodiment, carnauba wax is used to provide the extended release of the active ingredient.

The above-mentioned waxes can be used either alone or in combination. As known in the art, the following criteria may provide some guidance on deciding the ratio of wax and the active ingredient: if the proportion of the wax is too small, no satisfactory wax matrix may be obtained to provide the extended release. On the other hand, if the proportion of the wax is too large, formation of a wax matrix may not be adversely affected but the final dosage form, for instance, may become too bulky for oral intake.

The amount of the wax-based extended release material according to the present invention is preferably from about 1% to about 50% by weight of the total weight of granules, preferably from about 5% to about 25% by weight, and more preferably from about 8% to about 16% by weight.

In addition to an active ingredient and a wax-based extended release material, the formulation of the present invention may optionally contain various other inert additives, such as fillers, solvents, extenders, binders, wetting agents, plasticizers, disintegrators, surfactants and lubricants, such as defined in “Handbook of Pharmaceutical Excipients,” (American Pharmaceutical Association Pub., 3^{rd} Ed., 2000). The inert additive(s) may be added by methods known in the art, for example, by directly mixing with the wax-based extended release material and the active ingredient or adding while mixing the granulate containing the wax-based extended release material and the active ingredient. Preferably, the additives do not substantially alter the extended release provided by the wax-based extended release material.

Suitable plasticizers of the present invention include, but are not limited to, polyethylene glycols such as PEG300, PEG400, PEG600, PEG1450, PEG3350, PEG4600, and PEG800, stearic acid, propylene glycol, oleic acid, and triacetin.

Suitable wetting agents for use in conjunction with the present invention include polyethyleneglycols (PEGs) and their esters or ethers. One class of wetting agents which have little surfactant activity is the polyethylene glycol series, such as PEG 600. Other useful types in this series range in molecular weight from 200 to 7,000,000. In one embodiment of the invention, PEG 1450 and/or PEG 4600 are used.

Other suitable wetting agents include, but are not limited to, anionic surfactants such as sodium lauryl sulfate; sodium, potassium or magnesium n-dodecyl sulfate, n-tetradecylsulfate, n-hexadecyl sulfate, n-tetradecyloxyethyl sulfate, n-hexadecyloxyethyl sulfate or n-octadecyloxyethyl sulfate; or sodium, potassium or magnesium n-dodecanesulfonate; sodium, potassium or magnesium n-tetradecanesulfonate, n-hexadecanesulfonate or n-octadecanesulfonate, and the like. Many can also act as surface active agents.

The plasticizer may be added to the active ingredient first, or added to the wax-based extended release material first, or alternatively, to the blended mixture of the active ingredient and the wax-based extended release material.

Examples of suitable surfactants include, but are not limited to, non-ionic surfactants of the fatty acid polyhydroxy alcohol ester type such as sorbitan monolaurate, sorbitan monooleate, sorbitan monostearate or sorbitan monopalmitate, sorbitan tri-stearate or trioleate, polyethylene glycol fatty acid ester such as polyoxyethyl stearate, polyethylene glycol 600 stearate, and the like. Other examples of suitable surfactants include polyoxyethylene alkyl ethers, polyoxyethylene castor oil derivatives, sorbitan polyoxyethylene fatty acid esters, polyoxyethylene fatty acid esters, and polyoxyethylene stearates.

Examples of fillers include, but are not limited to, lactose anhydrous, lactose hydrous, microcrystalline cellulose, starch, pregelatinized starch, modified starch, dibasic calcium phosphate dihydrate, calcium sulfate trihydrate, calcium sulfate dihydrate, calcium carbonate, lactose, dextrose, sucrose, mannitol and sorbitol.

Examples of solvents include, but are not limited to, water, acetonitrile, ethyl acetate, acetone, benzene, toluene, dioxane, dimethylformamide, chloroform, methylene chloride, ethylene chloride, carbon tetrachloride, chlorobenzene, acetone, methanol, ethanol, isopropanol and butanol.

Examples of lubricants include, but are not limited to, magnesium stearate, calcium stearate, zinc stearate, talc, propylene glycol, PEG, stearic acid, vegetable oil, sodium benzoate, sodium lauryl sulfate, magnesium lauryl sulfate, mineral oil and polyoxyethylene monostearate.

Examples of binders include, but are not limited to, starches, e.g., potato starch, wheat starch, corn starch; gums, such as gum tragacanth, acacia gum and gelatin; microcrystalline cellulose, e.g., products known under the registered trademarks Avicel, Filtrak, Heweten or Pharmacel, hydroxypropyl cellulose, hydroxyethyl cellulose and hydroxypropylmethyl cellulose; and polyvinyl pyrrolidone, e.g., Povidone.

The wax-based extended release material, and other optional inert additives, may be included in any amount effective to provide a final extended release product having acceptable bioavailability. Typically, an acceptable bioavailability for a formulation of the present invention is one with equal or more than about 80% bioavailability, preferably equal or more than about 90%, more preferably equal or more than about 95% bioavailability as compared to a commercially available dosage form or a USP dosage form.

The bioavailability of Class II compounds depends on the rate of dissolution (see http://www.fda.gov/cder/OPS/BCS_guidance.htm for drug bioavailability classification). An example of acceptable bioavailability for a carbamazepine formulation of the present invention is one with equal or more than about 80% bioavailability, preferably equal or more than about 90%, more preferably equal or more than about 95% bioavailability as Carbatrol®. Such bioavailability may be assessed by comparing the dissolution rate of the formulation with Carbatrol®, as discussed in the following Examples. The dissolution profile of the formulations may be measured using USP apparatus II at 50 rpm in 1,000 mL simulated gastric fluid at pH 6.8 and at 37° C., as described in the Examples.

Preferably, the extended release formulations have at least from about 60% to 75% (by wt) active ingredient released after 2 hours, at least about from about 75% to 90% (by wt) active ingredient released after 4 hours, at least about from about 85% to 100% (by wt) active ingredient released after 8 hours. For example, the extended release formulations according to the present invention have at least about 50% (by wt) active ingredient released after 2 hours, at least about 70% (by wt) active ingredient released after 4 hours, at least about 80% (by wt) active ingredient released after 8 hours.

In certain embodiments of the present invention where the medicament is carbamazepine, the wax-based extended release material may be used from about 1% to about 50% by weight of the total weight of the granules, preferably from about 5% to about 25% by weight, and more preferably from about 8% to about 16% by weight. The plasticizer may be included in an amount from about 2% to about 30% by weight of the total weight of the granules, preferably from about 5% to about 15% by weight, more preferably from about 5% to about 10% by weight.

The formulation containing a poorly water soluble active ingredient and a wax-based extended release material of the present invention may be prepared by generally all conventional processes, including, but not limited to, granulation, grinding, compression, casting in a mold, tableting under pressure, and the like.

Preferably, the extended release formulation is prepared by hot melt granulation. The ingredients (both active and inert) can be added in any order during the granulation process. Granulation can take place in any conventional manner to produce a blend. For example, it can be prepared using a jacketed bowl equipped with planetary mixer, or using a hot melt extruder or a fluid bed granulator coater, mixed in a shell blender, a V-shape blender, a double cone blender, a ribbon mixer, and the like.

In one embodiment, the process for production of the solid dosage form of the present invention is hot melt granulation using an oil or steam jacketed double planetary motion mixer, or using a hot melt extruder or a fluid bed granulator coater.

The temperature for granulation according to the present invention is selected according to, among other factors, the types of wax and active ingredient, optional inert additives used, as well as the instrument used to prepare the extended release formulation. Generally speaking, these temperatures can be set at levels higher than the melting point of the wax by about 5-30° C., preferably about 10-20° C. In one embodiment, the active ingredient, and optionally, other inert additives are added to the hot wax. Granules are formed when the wax-based extended release material solidifies on the cold materials as they are added. The temperature may be kept slightly elevated (e.g., about 5-30° C., preferably about 10-20° C. higher than the melting point of the wax).

As used herein, the term “average granule size” (i.e. average particle size) is used interchangeably with the term “median granule size.” For example, an average size of mesh 60, according to the present invention, refers to a plurality of granules that, when characterized by sieving analysis, about 50% of the granules are retained on a 60 mesh screen. Similarly, an average size of mesh 40 refers to a plurality of granules that, when characterized by sieving analysis, about 50% of the granules are retained on a 40 mesh screen. See U.S. Pharmacopeia (USP, 2000), p. 1969 for correlations between mesh sizes and particle sizes in microns.

The desired average granule size may be achieved by any conventional means, for example, by passing the granules through a comill with a defined size screen. In one embodiment, the granules containing the active ingredient and the wax-based extended release material passed through a comill with 0.117″ round home screen, a comill with a 0.079″ grating screen, and/or a comill with a 0.045″ round hole screen. Particle sizes of the granules may be measured by a sieving method, for example, on an ATM Corporation Sonic Sifter. The screens that may be used include: 12, 20, 40, 60, 80, 100 (Mesh #). In one embodiment, the amplitude for sieving is set at 2 and tests are run for 5 minutes.

The average size of the granules may range from about 12 mesh to about 200 mesh, preferably from about 16 mesh to about 80 mesh and more preferably from about 16 mesh to about 70 mesh. The average size of the granules should be such so as to provide a final extended release product having an acceptable bioavailability.

The extended release formulation of the present invention may contain granules such that from about 40% to about 80% of the granules, preferably from about 55% to about 75% of the granules, more preferably from about 60% to about 70% of the granules are retained on a 60 mesh screen, e.g., when measured by a sieving method described above.

Methods for measuring and calculating average particle sizes are also known in the art, e.g., as described in Wadke & Jacobson, “Preformulation Testing,” in H. A. Lieberman & L. Lachman (Eds.), Pharmaceutical Dosage Forms: Tablets (1980), vol. 1, pp 1-10.

The formulation of the present invention can utilize any suitable dosage unit form, for example, tablets, tablets which disintegrate into granules, capsules, microcapsules or any other means which allow for oral administration. These forms may optionally be coated with pharmaceutically acceptable coating which disintegrates in the digestive system. Such coating may comprise a biodegradable polymer, a coloring and/or flavoring agent or any other suitable coating. The technique of preparing coated tablet, microcapsule or capsule formulations are known in the pharmaceutical sciences.

The amount of the active ingredient (i.e., drug) in the formulation varies depending on the desired dose for efficient drug delivery. The actual amount of the used drug is dependent on the patient's age, weight, sex, medical condition, disease or any other medical criteria. The actual drug amount is determined according to intended medical use by techniques known in the art. The pharmaceutical dosage formulated according to the invention may be administered once or more times per day, as determined by the attending physician.

For formulations where carbamazepine is the active ingredient, carbamazepine is typically formulated in amounts of about 0.001 to about 1.2 grams, preferably from about 0.2 to about 0.8 grams. The daily dose can be formulated in a single unit form or more than one unit, depending on the daily dose of carbamazepine and the number of times the formulation is to be administered. Carbamazepine, formulated according to the invention, may be delivered once or twice a day, depending on the release kinetics from the formulation. Carbamazepine formulations may be administered to fed or fasting patients.

In-vivo pharmacokinetic parameters (e.g., Cₘₐₓ and Tₘₐₓ) may be measured by methods known in the art. For carbamazepine formulations, this can be done by measuring the level of carbamazepine or its active metabolite, 10,11-epoxide, in the plasma, such as described in Mahmood and Chamberlin (1998, Br. J. Clin. Pharmacol. 45(3):241-6).

In one embodiment, a carbamazepine formulation, as measured by 10,11-epoxide plasma level, has a Tₘₐₓ of at least 24 hours, preferably at least 30 hours, more preferably at least 36 hours, or more. The carbamazepine formulation has a Cₘₐₓ of at least 0.10 μ/mL, preferably at least 0.12 μ/mL, more preferably at least 0.14 μ/mL, or more.

In another embodiment, a carbamazepine formulation, as measured by carbamazepine plasma level, has a Tₘₐₓ of at least 12 hours, preferably at least 15 hours, more preferably at least 20 hours, or more. The carbamazepine formulation has a Cₘₐₓ of at least 1.8 μ/mL, preferably at least 2.0 μ/mL, more preferably at least 2.5 μ/mL, or more.

The invention will be better understood by reference to the following examples which serve to illustrate but not to limit the present invention.

### EXAMPLE 1

### Preparation of Carbamazepine Capsules by Hot Melt Granulation

Formulation I

Ingredients include 300 mg carbamazepine, 54.0 mg Carnauba wax, 17.0 mg PEG 1450, 10.0 mg PEG 4600, and 44.0 mg lactose monohydrate. Formulations containing 200 mg or 100 mg carbamazepine contain the wax and other inert additives in amounts proportional to carbamazepine (same for the following examples).

Formulation II

Ingredients include 300 mg carbamazepine, 32.3 mg Carnauba wax, 10.2 mg PEG 1450, 5.94 mg PEG 4600, 23.74 mg lactose hydrous, and 2.84 mg lactose anhydrous.

Alternatively, ingredients include 300 mg carbamazepine, 32.3 mg Carnauba wax, 16.14 mg PEG 1450 or PEG 4600, and 26.58 mg lactose hydrous or lactose anhydrous.

Formulation III

Ingredients include 300 mg carbamazepine, 64.6 mg Carnauba wax, 20.4 mg PEG 1450, 11.88 mg PEG 4600, 47.48 mg lactose hydrous, and 5.68 mg lactose anhydrous.

Alternatively, ingredients include 300 mg carbamazepine, 64.6 mg Carnauba wax, 32.28 mg PEG 1450 or PEG 4600, and 53.16 mg lactose hydrous or lactose anhydrous.

Formulation IV

Ingredients include 300 mg carbamazepine, 54 mg Carnauba wax, 17 mg PEG 1450, 10 mg PEG 4600, and 44 mg lactose hydrous.

Alternatively, ingredients include 300 mg carbamazepine, 54 mg Carnauba wax, 27 mg PEG 1450 or PEG 4600, and 44 mg lactose hydrous.

Process I

Carnauba wax and both PEGs were added to in an oil or steam jacketed double planetary motion mixer and melted. A mixture of carbamazepine and lactose monohydrate was passed through a comill, and slowly added to the wax/PEG melt while a product temperature was maintained between 80° C.-90° C. and then heated to 95° C. The temperature may cool while mixing. The granulation was passed through a comill with a 0.117″ round hole screen, followed by a comill with a 0.079″ grating screen and subsequent with a 0.045 round hole screen. The granulation was blended in a V-blender for 5 minutes before encapsulation and packaging. The screens utilized were (Mesh #): 12, 20, 40, 60, 80, 100. An ATM Corporation Sonic Sifter was used with Amplitude set at 2 and the tests were run for 5 minutes.

Process II

Carnauba wax was added to a jacketed bowl with mixer, and melted at about 100° C., followed by the addition of both PEGs. Carbamazepine and one or both lactose ingredients were mixed thoroughly. The carbamazepine-lactose mixture was added incrementally to wax-PEG mixture and mixed until a uniform mixture was formed and cooled down to room temperature.

Particle size distributions for four carbamazepine batches of formulation I prepared according to Process I were determined by sieving, which was performed on an ATM Corporation Sonic Sifter. The results are summarized in Table 1.

TABLE 1

| | Formulation | | | |
|---|---|---|---|---|
| Screen | 1 | Formulation 2 | Formulation 3 | Formulation 4 |
| D&C | 0.18 | 0.00 | 0.01 | 0 |
| 12 | 0.14 | 0.15 | 0.18 | 0.06 |
| 20 | 5.14 | 6.89 | 16.05 | 4.2 |
| 40 | 32.84 | 40.16 | 40.69 | 25.34 |
| 60 | 24.23 | 19.03 | 17.54 | 23.7 |
| 80 | 9.81 | 9.06 | 7.29 | 13.25 |
| 100 | 5.36 | 4.22 | 3.27 | 5.74 |
| Fines | 22.30 | 20.49 | 14.97 | 27.71 |

### EXAMPLE 2

### Dissolution Testing

The dissolution profiles of carbamazepine Formulation I prepared according to Process I were measured according to the following method.

Discussion Testing Method

The dissolution test was performed at 37° C. in a USP apparatus II (paddles) at 50 rpm in 750 mL dissolution medium (pH 1.2), i.e., simulated gastric fluid (SGF), for 0-2 hours (acid phase), followed by adjustment to 1000 mL dissolution medium (pH 6.8) using phosphate buffer for 3-12 hours (basic phase). The SGF contains 0.9% sodium lauryl sulfate (SLS) but no enzymes. Samples were taken at hour 1 and 2 during the Acid Phase, and then at hour 3, 4, 5, 6, 8, and 12 during the Basic Phase.

Specifically, 750 mL of SGF without enzymes and 0.9% SLS were placed in each vessel and assemble apparatus. The above dissolution medium was allowed to equilibrate at 37° C. +/−0.5° C. Carbamazepine capsules were placed in each vessels, covered and placed in the USP apparatus for two hours (Acid Phase). Samples were withdrawn at the specified time for HPLC analysis. With the apparatus operating at the specified rate, 200 mL of 0.2M tribasic sodium phosphate that has been equilibrated to about 37° C. was added to each vessel. Deionized water was then added to each vessel for a final volume of 1000 mL. pH was adjusted to 6.8+/−0.05 using phosphoric acid or sodium hydroxide, if necessary. Dissolution was analyzed for an additional 10 hours (basic phase).

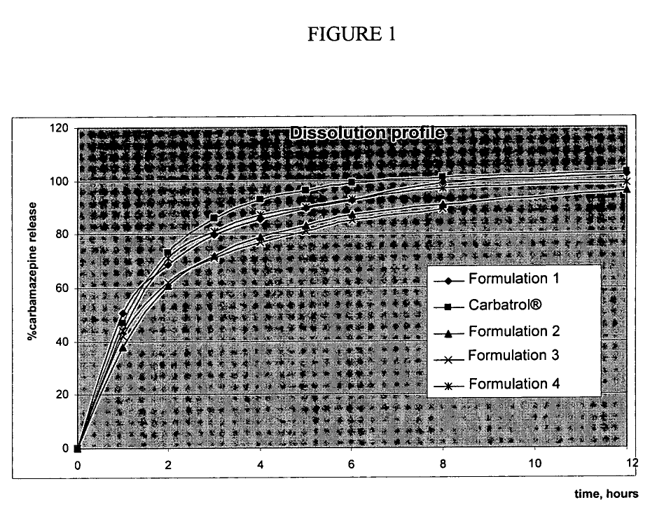

The dissolution profiles of the four batches, as measured using the foregoing protocol, are shown in FIG. 1. These results indicate that the carbamazepine formulation batches have comparable dissolution profiles as Carbatrol®.

### EXAMPLE 3

### Preparation of Indomethacisn 75 mg Capsules by Hot Melt Granulation

Formulation

Ingredients can include 75 mg indomethacin, 32.3 mg Carnauba wax, 10.2 mg PEG 1450, 5.94 mg PEG 4600, 76.58 mg lactose hydrous or lactose anhydrous.

Alternatively, ingredients can include 75 mg indomethacin, 32.3 mg Bees wax, 16.14 mg PEG 1450 or PEG 4600, and 76.58 mg lactose hydrous or lactose anhydrous.

Alternatively, ingredients include 75 mg indomethacin, 64.6 mg Bees wax, 20.4 mg PEG 1450 or PEG 4600, and 40 mg lactose hydrous or lactose anhydrous.

Process

Carnauba wax or bees wax is added to a jacketed bowl with mixer, and is melted at about 1 00° C., followed by the addition of both PEGs. Indomethacin and one or both lactose ingredients are mixed thoroughly. The indomethacin-lactose mixture is added incrementally to wax-PEG mixture and mixed until a uniform mixture is formed and cooled down to room temperature. Particle sizes of the granules can be characterized as described in Example 1.

### EXAMPLE 4

### Preparation of Ketoprofen 200 mg Capsules by Hot Melt Granulation Formulation

Ingredients can include 200 mg ketoprofen, 32.3 mg Carnauba wax, 10.2 mg PEG 1450, 5.94 mg PEG 4600, and 51.56 mg lactose hydrous or lactose anhydrous.

Alternatively, ingredients can include 200 mg ketoprophen, 32.3 mg Bees wax, 16.14 mg PEG 1450 or PEG 4600, and 51.56 mg lactose hydrous or lactose anhydrous.

Alternatively, ingredients can include 200 mg ketoprophen, 64.6 mg Bees wax, 20.4 mg PEG 1450 or PEG 4600, and 15 mg lactose hydrous or lactose anhydrous.

Process

Carnauba wax or bees wax is added to a jacketed bowl with mixer, and is melted at about 100° C., followed by the addition of both PEGs. Ketoprophen and one or both lactose ingredients are mixed thoroughly. The ketoprophen-lactose mixture is added incrementally to wax-PEG mixture and mixed until a uniform mixture is formed and cooled down to room temperature. Particle sizes of the granules can be characterized as described in Example 1.

### EXAMPLE 5

### Preparation of Phendimetrazine Tartrate Capsules by Hot Melt Granulation

Formulation

Ingredients can include 105 mg Phendimetrazine tartrate, 52.3 mg white wax, 10.2 mg PEG 1450, 5.94 mg PEG 4600, and 76.56 mg lactose hydrous or lactose anhydrous.

Alternatively, ingredients can include 105 mg Phendimetrazine tartrate, 52.3 mg stearyl alcohol, 16.14 mg PEG 1450 or PEG 4600, and 76.56 mg lactose hydrous or lactose anhydrous.

Alternatively, ingredients can include 105 mg Phendimetrazine tartrate, 52.3 mg Bees wax, 20.4 mg PEG 1450 or PEG 4600, and 72.3 mg lactose hydrous or lactose anhydrous.

Process

Carnauba wax or bees wax is added to a jacketed bowl with mixer, and is melted at about 100° C., followed by the addition of both PEGs. Phendimetrazine tartrate and one or both lactose ingredients are mixed thoroughly. The Phendimetrazine tartrate-lactose mixture is added incrementally to wax-PEG mixture and mixed until a uniform mixture is formed and cooled down to room temperature. Particle sizes of the granules can be characterized as described in Example 1.

Equivalents

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

### INCORPORATION BY REFERENCE

All publications and patent documents cited in this application are incorporated by reference in their entirety for all purposes to the same extent as if the entire contents of each individual publication or patent document was incorporated herein.

What is claimed is:

1. An extended release formulation comprising a plurality of granules comprising a poorly water soluble active ingredient and a wax-based extended release material, wherein the plurality of granules, when characterized using sieve analysis, have an average size from about 10 mesh to about 100 mesh.

2. The formulation of claim 1, wherein the granules have an average size from about 10 mesh to about 80 mesh.

3. The formulation of claim 2, wherein the granules have an average particle size from about 16 mesh to about 70 mesh.

4. An extended release formulation comprising a plurality of granules comprising a poorly water soluble active ingredient and a wax-based extended release material, such that, when characterized by sieve analysis, about 40% to 80% of the granules are retained on a 60 mesh screen.

5. The extended release formulation of claim 4, wherein about 55% to about 75% of the granules are retained on a 60 mesh screen.

6. The extended release formulation of claim 5, wherein about 60% to about 70% of the granules are retained on a 60 mesh screen.

7. The formulation of claim 1 or 4, wherein the active ingredient is selected from the group consisting of carbamazepine, phendimetrazine tartrate, indomethacin, disopyramide phosphate, and ketoprofen.

8. The formulation of claim 1 or 4, wherein the active ingredient is carbamazepine.

9. The formulation of claim 1 or 4, wherein the wax-based extended release material is carnauba wax, bees wax or a combination thereof.

10. The formulation of claim 1 or 4, wherein the wax-based extended release material is present in an amount from about 1% to about 50% by weight of total weight of the granules.

11. The formulation of claim 10, wherein the wax-based extended release material is present in an amount from about 5% to about 25% by weight of total weight of the granules.

12. The formulation of claim 11, wherein the wax-based extended release material is present in an amount from about 8% to about 16% by weight of total weight of the granules.

13. The formulation of claim 1 or 4, further comprising one or more inert additives selected from the group consisting of a wetting agent, a filler, a binder, and a surfactant.

14. An extended release formulation comprising a plurality of granules comprising carbamazepine and a wax-based extended release material, wherein the extended release formulation has an in vitro dissolution profile, when measured using a USP apparatus II at 50 rpm in 1,000 mL dissolution medium (pH 1.2 and then pH 6.8) at 37° C., such that from about 60% to about 75% (by wt) active ingredient is released after 2 hours, from about 75% to about 90% (by wt) active ingredient is released after 4 hours, from about 85% to about 100% (by wt) active ingredient is released after 8 hours.

15. The extended release formulation of claim 14, wherein the in vitro release profile is chosen such that the peak plasma level of carbamazepine obtained in vivo occurs at least 15 hours after administration.

16. A method for preparing an extended release formulation containing a poorly water soluble active ingredient, said method comprising:
(a) melting a wax-based extended release material; and
(b) mixing the active ingredient with the melted wax-based extended release material at a temperature higher than the melting temperature of the wax-based extended release material to produce the extended release formulation.

17. The method of claim 16, further comprising the step of (c) performing sieve analysis to select granules having an average size from about 10 mesh to about 100 mesh to produce the extended release formulation.

18. The method of claim 17, wherein granules are selected to have an average size from about 10 mesh to about 80 mesh.

19. The method of claim 18, wherein granules are selected to have an average size from about 16 mesh to about 70 mesh.

20. The method of claim 16, further comprising the step of (c) performing sieve analysis to select granules such that from about 40% to about 80% of the granules are retained on a 60 mesh sieve to produce the extended release formulation.

21. The method of claim 20, wherein granules are selected such that from about 55% to about 75% of the granules are retained on a 60 mesh sieve.

22. The method of claim 21, wherein granules are selected such that from about 60% to about 70% of the granules are retained on a 60 mesh sieve.

23. The method of claim 16, wherein the active ingredient is carbamazepine.

24. The method of claim 16, comprising mixing one or more inert additives with the melted wax-based extended release material.
